(19) United States
(12) Patent Application Publication
(10) Pub. No.: US 2012/0245188 A1
(43) Pub. Date: Sep. 27, 2012

(54) METHODS OF TREATING MEMORY LOSS AND ENHANCING MEMORY PERFORMANCE

(75) Inventors: Matthew Huentelman, Phoenix, AZ (US); Heather Bimonte-Nelson, Phoenix, AZ (US)

(73) Assignee: TRANSLATIONAL GENOMICS RESEARCH INSTITUTE, Phoenix, AZ (US)

(21) Appl. No.: 13/297,149

(22) Filed: Nov. 15, 2011

Related U.S. Application Data
(60) Provisional application No. 61/413,572, filed on Nov. 15, 2010.

(51) Int. Cl. A61K 31/506; A61P 25/32; A61P 25/16; A61P 25/00; A61P 25/28

(52) U.S. Cl. 514/274

(57) ABSTRACT

The present invention provides methods and compositions for enhancing working memory impaired due to aging, Alzheimer's disease, amyotrophic lateral sclerosis (ALS), Parkinson's disease, alcoholism or alcohol withdrawal or Huntington's disease using p38 MAPK inhibitor such as SB239063.

### CROSS REFERENCE

This application is related to and claims the priority benefit of U.S. provisional application 61/413,572 entitled COMPOSITIONS USEFUL IN THE CONTROL OF MEMORY, filed on Nov. 15, 2010, the teachings and content of which are incorporated by reference herein.

### STATEMENT REGARDING FEDERALLY SPONSORED RESEARCH

The U.S. government retains certain rights in this invention as provided by the terms of Grant Number R01 NS059873, awarded by the National Institutes of Health.

### FIELD OF THE INVENTION

The present invention is related to methods and compositions for treatment of short term memory impairment relating to conditions such as aging, Alzheimer's disease, amyotrophic lateral sclerosis (ALS), Parkinson's disease, alcoholism or alcohol withdrawal, or Huntington's disease in which p38 mitogen activated protein kinase (MAPK) is activated. Specifically, the invention is related to short term memory enhancement using p38 MAPK inhibitor.

### BACKGROUND OF THE INVENTION

Alzheimer's disease (AD) is the most common cause of disabling memory and thinking problems in older persons. According to one study, it afflicts about 10% of those over the age of 65 and almost half of those over the age of 85. According to another study, the prevalence of the disorder increases from 1% by the age of 60 years to 40% in nonagenarians. By 2050, the number of afflicted persons is projected to quadruple, leading to approximately 16 million patients and a cost of more than $750 billion per year in the United States alone. In the meantime, the disorder takes a devastating toll on patients and their families. Clinically, AD is characterized by gradual but progressive declines in memory, language skills, the ability to recognize objects or familiar faces, the ability to perform routine tasks, and judgment and reasoning. Associated features commonly include agitation, paranoid delusions, sleepiness, aggressive behaviors, and wandering. In its most severe form, patients may be confused, bed-ridden, unable to control their bladder or bowel functions, or swallow. By contributing to other problems (e.g., inanition and infections), it is considered the fourth leading cause of death in the United States. Neuropathologically, AD is characterized by the accumulation of neuritic plaques (the major component of which is the amyloid-B peptide [Aβ]), neurofibrillary tangles (NFT, the major component of which is the hyper-phosphorylated form of the protein tau). While the etiology leading to the development of AD has not been clearly resolved, genetic factors play a major role. Therefore, methods and compositions for improving AD signs and symptoms including impaired memory are needed.

Although the neuropathological features of AD have been well defined, the underlying mechanisms responsible for the pathogenic process have not been clearly delineated. Among the hypotheses that have been proposed to explain the pathogenesis of AD is the concept of inflammatory and oxidative stress, now accepted as components of the pathology of AD. Oxidative stress has been shown to contribute to the neuropathology of a number of neurodegenerative disorders including Alzheimer's disease. This is further implicated in neuronal loss, associated with age-related cognitive decline and neuro inflammation. Many stress stimuli have been demonstrated to be converted into specific cellular responses through the activation of mitogen activated protein kinase (MAPK) signaling pathways. Oxidative stress and inflammation seems to be the major stimuli for MAPK signaling cascades with cell survival or death as a possible consequence. After being phosphorylated by upstream kinases, MAPKs become active serine/threonine protein kinases that can phosphorylate both cytoplasmic and nuclear targets. Among diverse MAPK family members, p38 MAPK subfamilies are preferentially activated by cellular stresses, such as those induced by proinflammatory cytokines and oxidative stress. Aberrant activation of p38 MAPK has also been implicated in the pathogenesis of Alzheimer's disease including: neuroinflammation oxidative stress amyloid beta production tau tangle formation and neuronal degeneration. Therefore, p38 MAPK may be a target for addressing memory impairment associated with inflammatory or oxidative stress that activates MAPK signaling pathways.

### SUMMARY OF THE INVENTION

Provided herein is a method of treating short term memory impairment comprising the step of: administering a therapeutically effective dose of a pharmaceutical composition to a subject, wherein said pharmaceutical composition comprises at least one pharmaceutically acceptable carrier and a p38 mitogen activated protein kinase (MAPK) inhibitor. In the general method, the p38 MAPK inhibitor comprises a molecule structure or a derivative thereof as follows:

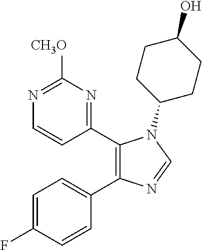

The provided method is applicable to short term memory impairment that is related to one or more conditions selected from the group consisting of aging, Alzheimer's disease, amyotrophic lateral sclerosis (ALS), Parkinson's disease, alcoholism or alcohol withdrawal and Huntington's disease. In one example, the short term memory impairment is related to Alzheimer's disease. In another example, the short term memory impairment is related to Parkinson's disease. In one application of the method for enhancing short term memory performance, the effective dose of the pharmaceutical composition comprising a p38 MAPK inhibitor is at a range between about 0 to 30 mg/kg (body weight), preferably between 10 to 20 mg/kg, and more preferably 15 mg/kg, and this p38 MAPK inhibitor comprises a molecule having a structure or a derivative thereof as follows:

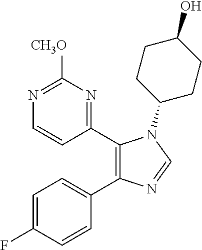

In another application of the method for improving short term memory, the effective dose of the pharmaceutical composition comprising a p38 MAPK inhibitor is at a range between about 0 and 50 mg/kg, preferably between about 10 and 40 mg/kg, more preferably at 30 mg/kg; and the p38 MAPK inhibitor comprises a molecule having a structure or a derivative thereof as follows:

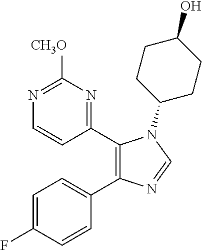

The general method may further comprise the step of administering a second treatment modality to the subject, which may comprise administering a second pharmaceutical composition to the subject. In one application, the second pharmaceutical composition is administered concurrently with the first pharmaceutical composition.

Also provided herein is a method of enhancing spatial reference memory performance, and the method comprises the step of administering a therapeutically effective dose of a pharmaceutical composition to a subject, wherein said pharmaceutical composition comprises at least one pharmaceutically acceptable carrier and a p38 mitogen activated protein kinase (MAPK) inhibitor. In one example, the p38 MAPK inhibitor comprises a molecule having a structure or a derivative thereof as follows:

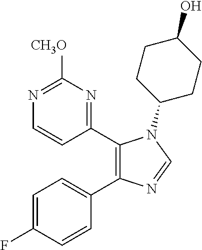

The spatial reference memory impairment to be treated under the method may be related to one or more conditions selected from the group consisting of aging, Alzheimer's disease, amyotrophic lateral sclerosis (ALS), Parkinson's disease, alcoholism or alcohol withdrawal and Huntington's disease. In one example, the spatial reference memory impairment is related to Alzheimer' s disease. In another example, the spatial reference memory impairment is related to Alzheimer's disease is Parkinson's disease. In one application, the effective dose of the pharmaceutical composition comprising a p38 MAPK inhibitor for treating impaired spatial reference memory is at a range between about 0 and 50 mg/kg, preferably between about 10 and 40 mg/kg, more preferably at 30 mg/kg, and such p38 MAPK inhibitor comprises a molecule having a structure or a derivative thereof as follows:

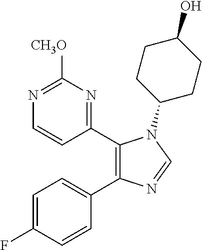

The method of treating impaired spatial reference memory may further comprise the step of administering additional or a second treatment modality to the subject. Said additional or second treatment modality may comprise administering a second pharmaceutical composition to the subject. In one example, the second pharmaceutical composition may be administered concurrently with the first pharmaceutical composition.

Other aspects and iterations of the invention are described in more detail below.

### BRIEF DESCRIPTION OF THE FIGURES

FIG. 1 depicts the study design relating to testing groups of the rat, drug delivery route, vehicle, doses and memory testing systems;

FIG. 2 depicts the Morris Water Maze and the Delayed Match to Sample Water Maze, and the test procedure using the mazes;

FIG. 3 depicts the effect of SB239063 [trans-1-(4-hydroxycyclohexyl)-4-(4-fluorophenyl)-5-(2-methoxypyridimidin-4-yl)imidazole] on spatial reference memory by comparing swim distance (cm) across all testing days using Morris Water Maze among groups of rats treated with vehicle only, low dose of SB239063 at 15 mg/kg or high dose of SB239063 at 30 mg/kg; and

FIG. 4A depicts the effect of SB239063 on working spatial reference memory by comparing Three Choice Task errors across all trials in days 1-9 using Delay Match To Sample Water Maze among groups of rats treated with vehicle only, low dose of SB239063 at 15 mg/kg and high dose of SB239063 at 30 mg/kg. FIG. 4B depicts the Three Choice Task errors on trial 3 among groups of rats treated with vehicle only, low dose of SB239063 at 15 mg/kg and high dose of SB239063 at 30 mg/kg.

### DETAILED DESCRIPTION OF THE INVENTION

The present invention provides methods and compositions for treating memory impairment associated with mammalian p38 mitogen-activated protein kinases (MAPKs). The mitogen-activated protein (MAP) p38 kinase is a ubiquitous and highly conserved, proline-directed serine-threonine protein kinase. p38 MAPKs can be activated by various cellular stresses and appears to play an important role in a variety of pathophysiological responses, which have been suggested to be involved in many processes considered critical to the inflammatory response, apoptosis, tissue growth or differentiation. Several of these events are hallmarks of pulmonary diseases such as chronic obstructive pulmonary disease and asthma. In the central nervous systems (CNS), activation of the p38 MAPK pathway constitutes a key step in the development of several diseases, and the molecular mechanisms mediated by p38 MAPK signaling have been defined. Activation of this cascade releases pro-inflammatory cytokines that are known to be involved in cerebral ischemia, Alzheimer's disease (AD), Parkinson's disease (PD), multiple sclerosis (MS), neuropathic pain and depression. In AD, stimulated p38 MAPK may trigger the hyperphosphorylation of a neural microtubule-associated protein, tau.

Inhibition of p38 MAPK has been reported to reduce oxidative damage and neuroinflammatory events in various experimental models. The present invention provides methods and compositions for treating memory impairment using a p38 MAPK inhibitor, SB 239063 [trans-1-(4-hydroxycyclohexyl)-4-(4-fluorophenyl)-5-(2-methoxypyridimidin-4-yl)imidazole, which inhibits both α and β forms of p38 MAPK.

### (I) Pharmaceutical Composition

SB 239063 [trans-1-(4-hydroxycyclohexyl)-4-(4-fluorophenyl)-5-(2-methoxypyridimidin-4-yl)imidazole] is a p38 mitogen-activated protein kinase selective inhibitor with known anti-inflammatory/antiallergic activity and with potential utility for the treatment of asthma and other inflammatory disorders. When administered orally, SB 239063 inhibited lipopolysaccharide-induced plasma tumor necrosis factor production (IC₅₀=2.6 mg/kg) and reduced adjuvant-induced arthritis (51% at 10 mg/kg) in rats. SB 239063 reduced infarct volume (48%) and neurological deficits (42%) with oral administeration of 15 mg/kg before moderate stroke. SB 239063 was also reported to protect against mild excitotoxic neuronal injury caused by NMDA (N-methyl D-aspartate) and provide substantial protection against cell death induced by either oxygen glucose deprivation (OGD) or magnesium deprivation in cultured neurons. Excitotoxicity may be involved in a wide range of conditions including, but not limited to, spinal cord injury, stroke, traumatic brain injury, hearing loss (through noise overexposure or ototoxicity) and in neurodegenerative diseases of the central nervous system (CNS) such as multiple sclerosis, Alzheimer's disease, amyotrophic lateral sclerosis (ALS), Parkinson's disease, alcoholism or alcohol withdrawal, and Huntington's disease. Other common conditions that cause excessive glutamate concentrations around neurons are hypoglycemia and status epilepticus.

One aspect of the invention provides using SB 239063 [trans-1-(4-hydroxycyclohexyl)-4-(4-fluorophenyl)-5-(2-methoxypyridimidin-4-yl)imidazole] and derivatives thereof to enhance memory which is impaired due to aging or medical conditions, such as, AD. The provided pharmaceutical composition comprises a compound with the following structure:

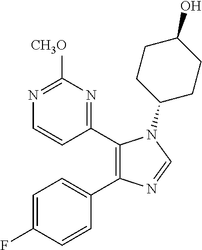

The disclosed compound and its intermediates may exist in different tautomeric forms. Tautomers include any structural isomers of different energies that have a low energy barrier to interconversion. One example is proton tautomers (prototropic tautomers.) In this example, the interconversions occur via the migration of a proton. Examples of prototropic tautomers include, but are not limited to keto-enol and imine-enamine isomerizations. In another example illustrated graphically below, proton migration between the 1-position and 3-position nitrogen atoms of the benzimidazole ring may occur. As a result, Formulas Ia and Ib are tautomeric forms of each other:

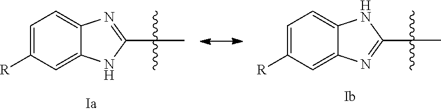

The disclosed compound further encompasses any other physiochemical or sterochemical form that the disclosed compound may assume. Such forms include diastereomers, racemates, isolated enantiomers, hydrated forms, solvated forms, or any other known or yet to be disclosed crystalline, polymorphic crystalline, or amorphous form. Amorphous forms lack a distinguishable crystal lattice and therefore lack an orderly arrangement of structural units. Many pharmaceutical compounds have amorphous forms. Methods of generating such chemical forms will be well known by one with skill in the art.

Such pharmaceutical compositions may take any physical form necessary depending on a number of factors including the desired method of administration and the physicochemical and stereochemical form taken by the compound or pharmaceutically acceptable salts of the compound. Such physical forms include a solid, liquid, gas, sol, gel, aerosol, or any other physical form now known or yet to be disclosed.

The concept of a pharmaceutical composition including the disclosed compound also encompasses the disclosed compound or a pharmaceutically acceptable salt thereof with or without any other additive. The physical form of the invention may affect the route of administration and one skilled in the art would know to choose a route of administration that takes into consideration both the physical form of the compound and the disorder to be treated. Pharmaceutical compositions that include the disclosed compound may be prepared using methodology well known in the pharmaceutical art. A pharmaceutical composition that includes the disclosed compound may include a second effective compound of a distinct chemical formula from the disclosed compound. This second effective compound may have the same or a similar molecular target as the target or it may act upstream or downstream of the molecular target of the disclosed compound with regard to one or more biochemical pathways.

Pharmaceutical compositions, including the disclosed compound include materials capable of modifying the physical form of a dosage unit. In one nonlimiting example, the composition includes a material that forms a coating that contains the compound. Materials that may be used in a coating, include, for example, sugar, shellac, gelatin, or any other inert coating agent.

Pharmaceutical compositions including the disclosed compound may be prepared as a gas or aerosol. Aerosols encompass a variety of systems including colloids and pressurized packages. Delivery of a composition in this form may include propulsion of a pharmaceutical composition including the disclosed compound through use of liquefied gas or other compressed gas or by a suitable pump system. Aerosols may be delivered in single phase, bi-phasic, or multi- phasic systems.

In some aspects of the invention, the pharmaceutical composition including the disclosed compound is in the form of a solvate. Such solvates are produced by the dissolution of the disclosed compound in a pharmaceutically acceptable solvent. Pharmaceutically acceptable solvents include any mixtures of one or more solvents. Such solvents may include pyridine, chloroform, propan-1-ol, ethyl oleate, ethyl lactate, ethylene oxide, water, ethanol, and any other solvent that delivers a sufficient quantity of the disclosed compound to treat the indicated condition.

Pharmaceutical compositions that include the disclosed compound may also include at least one pharmaceutically acceptable carrier. Carriers include any substance that may be administered with the disclosed compound with the intended purpose of facilitating, assisting, or helping the administration or other delivery of the compound. Carriers include any liquid, solid, semisolid, gel, aerosol or anything else that may be combined with the disclosed compound to aid in its administration. Examples include diluents, adjuvants, excipients, water, and oils (including petroleum, animal, vegetable or synthetic oils.) Such carriers include particulates such as a tablet or powder, liquids such as oral syrup or injectable liquid, and inhalable aerosols. Further examples include saline, gum acacia, gelatin, starch paste, talc, keratin, colloidal silica, and urea. Such carriers may further include binders such as ethyl cellulose, carboxymethylcellulose, microcrystalline cellulose, or gelatin; excipients such as starch, lactose or dextrins; disintegrating agents such as alginic acid, sodium alginate, Primogel, and corn starch; lubricants such as magnesium stearate or Sterotex; glidants such as colloidal silicon dioxide; sweetening agents such as sucrose or saccharin, a flavoring agent such as peppermint, methyl salicylate or orange flavoring, or coloring agents. Further examples of carriers include polyethylene glycol, cyclodextrin, oils, or any other similar liquid carrier that may be formulated into a capsule. Still further examples of carriers include sterile diluents such as water for injection, saline solution, physiological saline, Ringer's solution, isotonic sodium chloride, fixed oils such as synthetic mono or digylcerides, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose, thickening agents, lubricating agents, and coloring agents.

The pharmaceutical composition, including the disclosed compound, may take any of a number of formulations depending on the physicochemical form of the composition and the type of administration. Such forms include solutions, suspensions, emulsions, tablets, pills, pellets, capsules, capsules including liquids, powders, sustained-release formulations, directed release formulations, lyophylates, suppositories, emulsions, aerosols, sprays, granules, powders, syrups, elixirs, or any other formulation now known or yet to be disclosed. Additional examples of suitable pharmaceutical carriers and formulations are well known in the art.

Methods of administration include, but are not limited to, oral administration and parenteral administration. Parenteral administration includes, but is not limited to intradermal, intramuscular, intraperitoneal, intravenous, subcutaneous, intranasal, epidural, sublingual, intramsal, intracerebral, iratraventricular, intrathecal, intravaginal, transdermal, rectal, by inhalation, or topically to the ears, nose, eyes, or skin. Other methods of administration include but are not limited to infusion techniques including infusion or bolus injection, by absorption through epithelial or mucocutaneous linings such as oral mucosa, rectal and intestinal mucosa. Compositions for parenteral administration may be enclosed in ampoule, a disposable syringe or a multiple-dose vial made of glass, plastic or other material.

Administration may be systemic or local. Local administration is administration of the disclosed compound to the area in need of treatment. Examples include local infusion during surgery; topical application, by local injection; by a catheter; by a suppository; or by an implant. Administration may be by direct injection into the central nervous system by any suitable route, including intraventricular and intrathecal injection. Intraventricular injection can be facilitated by an intraventricular catheter, for example, attached to a reservoir, such as an Ommaya reservoir. Pulmonary administration may be achieved by any of a number of methods known in the art. Examples include the use of an inhaler or nebulizer, formulation with an aerosolizing agent, or via perfusion in a fluorocarbon or synthetic pulmonary surfactant. The disclosed compound may be delivered in the context of a vesicle such as a liposome or any other natural or synthetic vesicle. Additional examples of suitable modes of administration are well known in the art.

A pharmaceutical composition formulated to be administered by injection may be prepared by dissolving the disclosed compound with water so as to form a solution. In addition, a surfactant may be added to facilitate the formation of a homogeneous solution or suspension.

Surfactants include any complex capable of non-covalent interaction with the disclosed compound so as to facilitate dissolution or homogeneous suspension of the compound.

Pharmaceutical compositions including the disclosed compound may be prepared in a form that facilitates topical or transdermal administration. Such preparations may be in the form of a solution, emulsion, ointment, gel base, transdermal patch or iontophoresis device. Examples of bases used in such compositions include opetrolatum, lanolin, polyethylene glycols, beeswax, mineral oil, diluents such as water and alcohol, and emulsifiers and stabilizers, thickening agents, or any other suitable base now known or yet to be disclosed.

Determination of an effective amount of the disclosed compound is within the capability of those skilled in the art, especially in light of the detailed disclosure provided herein. The effective amount of a pharmaceutical composition used to affect a particular purpose as well as its toxicity, excretion, and overall tolerance may be determined in vitro, or in vivo, by pharmaceutical and toxicological procedures either known now by those skilled in the art or by any similar method yet to be disclosed. One example is the in vitro determination of the IC₅₀ (half maximal inhibitory concentration) of the pharmaceutical composition in cell lines or target molecules. Another example is the in vivo determination of the LD₅₀ (lethal dose causing death in 50% of the tested animals) of the pharmaceutical composition. The exact techniques used in determining an effective amount will depend on factors such as the type and physical/chemical properties of the pharmaceutical composition, the property being tested, and whether the test is to be performed in vitro or in vivo. The determination of an effective amount of a pharmaceutical composition will be well known to one of skill in the art who will use data obtained from any tests in making that determination. Determination of an effective amount of disclosed compound for administration also includes the determination of an effective therapeutic amount and a pharmaceutically acceptable dose, including the formulation of an effective dose range for use in vivo, including in humans.

Treatment of a condition is the practice of any method, process, or procedure with the intent of halting, inhibiting, slowing or reversing the progression of a disease, disorder or condition, substantially ameliorating clinical symptoms of a disease disorder or condition, or substantially preventing the appearance of clinical symptoms of a disease, disorder or condition, up to and including returning the diseased entity to its condition prior to the development of the disease. Generally, the effectiveness of treatment is determined by comparing treated groups with non-treated groups.

The addition of a therapeutically effective amount of the disclosed compound encompasses any method of dosing of a compound. Dosing of the disclosed compound may include single or multiple administrations of any of a number of pharmaceutical compositions that include the disclosed compound as an active ingredient. Examples include a single administration of a slow release composition, a course of treatment involving several treatments on a regular or irregular basis, multiple administrations for a period of time until a diminution of the disease state is achieved, preventative treatments applied prior to the instigation of symptoms, or any other dosing regimen known in the art or yet to be disclosed that one skilled in the art would recognize as a potentially effective regimen. A dosing regimen including the regularity of and mode of administration will be dependent on any of a number of factors including but not limited to the subject being treated; the severity of the condition; the manner of administration, the stage of disease development, the presence of one or more other conditions such as pregnancy, infancy, or the presence of one or more additional diseases; or any other factor now known or yet to be disclosed that affects the choice of the mode of administration, the dose to be administered and the time period over which the dose is administered.

Pharmaceutical compositions that include the disclosed compound may be administered prior to, concurrently with, or after administration of additional or second pharmaceutical compositions that may or may not include the compound. Concurrent administration means compositions are administered within about one minute of each other. If not administered concurrently, the additional or second pharmaceutical compositions may be administered a period of one or more minutes, hours, days, weeks, or months before or after the pharmaceutical composition that includes the currently disclosed compound. Alternatively, a combination of pharmaceutical compositions may be cyclically administered. Cycling therapy involves the administration of one or more pharmaceutical compositions for a period of time, followed by the administration of one or more different pharmaceutical compositions for a period of time and repeating this sequential administration. Cycling therapy may be used, for example, to reduce the development of resistance to one or more of the compositions, to avoid or reduce the side effects of one or more of the compositions, and/or to improve the efficacy of the treatment.

The invention further encompasses kits that facilitate the administration of the disclosed compound to a diseased entity. An example of such a kit includes one or more unit dosages of the compound. The unit dosage would be enclosed in a preferably sterile container and would be comprised of the disclosed compound and a pharmaceutically acceptable carrier. In another aspect, the unit dosage would comprise one or more lyophilates of the compound. In this aspect of the invention, the kit may include another preferably sterile container enclosing a solution capable of dissolving the lyophilate. However, such a solution need not be included in the kit and may be obtained separately from the lyophilate. In another aspect, the kit may include one or more devices used in administrating the unit dosages or a pharmaceutical composition to be used in combination with the compound. Examples of such devices include, but are not limited to, a syringe, a drip bag, a patch or an enema. In some aspects of the invention, the device comprises the container that encloses the unit dosage. In another aspect, the kit may include one or more additional compounds for administration and administration instructions therefor.

Pharmaceutical compositions including the disclosed compound may be used in methods of treating memory loss or enhancing memory performance. Such methods involve the administration of an effective amount of a pharmaceutical composition that includes the disclosed compound and/or a pharmaceutically acceptable salt thereof to a mammal.

### (II) Method of Improving Memory Impairment Related to AD

Another aspect of the invention provides methods of enhancing working memory in a subject. The subjects for the provided method include but are not limited to mammals (in particular, humans).

Although long-term memory deficits are the hallmark of AD, deficits in short-term memory of information as well as higher level deficits result in AD patients related to the diminished ability to coordinate multiple tasks or to inhibit irrelevant information. Short-term memory is also referred to as working memory, primary memory, immediate memory, operant memory, or provisional memory. Short-term/working memory tasks are those that require the goal-oriented active monitoring or manipulation of information or behaviors in the face of interfering processes and distractions. Working memory can be divided into separate systems for retaining location information and object information (colors, shapes), which are commonly referred to as spatial working memory (SWM) and visual (or object) working memory (VWM), respectively. In one embodiment, the method provided improves the short-term memory in the AD patient such that the impairments in dual-task performance, inhibitory ability, and set-shifting ability are alleviated. In one embodiment, the method provided improves the short-term memory in the AD patient such that the ability to remember information over a brief period of time (in the order of seconds), and the ability to actively hold information in the mind needed to do complex tasks such as reasoning, comprehension and learning is improved.

The methods of improving working memory associated to an AD patient may comprise the step of testing the working memory capacity during and after the treatment. The working memory capacity can be tested by a variety of tasks. With animals, such as rats, mazes are commonly used to determine whether different treatments or conditions affect learning and memory in rats. For example, the Multiple T-maze, a complex maze made of many T-junctions, or the Y-maze with three identical arms, can be used to answer questions of place versus response learning and cognitive maps; can be used to answer questions of place versus response learning and cognitive maps. The radial arm maze, in general, having a center platform with eight, twelve, or sixteen spokes radiating out from a central core, can be used for testing short-term memory. To test this, a single food pellet is placed at the end of each arm. A rat is placed on the central platform. The rat visits each arm and eats the pellet. To successfully complete the maze, the rat must go down each arm only once. He must use short-term memory and spatial cues to remember which arms he's already visited. If a rat goes down an arm twice, this counts as an error. The rats might be given particular drugs or treatment conditions to see if these impair or enhance short-term memory. In one embodiment, the subject may be administered a pharmaceutical composition comprising a compound having a structure or derivative thereof as follows:

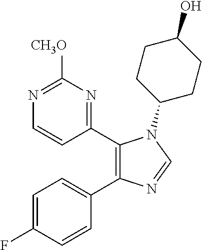

Working memory can also be tested using the Morris water maze. In general, the Morris water maze is a large round tub of opaque water with two small hidden platforms located 1-2 cm under the water's surface. The rat is placed on a start platform. The rat swims around until it finds the other platform to stand on. External cues, such as patterns or the standing researcher, are placed around the pool in the same spot every time to help the rat learn where the end platform is. The researcher measures how long it takes for a rat to find hidden platform, by changing or moving and using different spatial cues. The Morris water maze tests the spatial learning, cognitive maps and memory. The rats under the Morris water maze test may be given particular drugs or treatment conditions to see if these impair or enhance short-term memory. In one embodiment, the subject may be administered a pharmaceutical composition comprising SB 239063.

Other methods of evaluating spatial and visual working memory include Delayed-Match to Sample (DMS) asymmetrical 3-choice task, which is illustrated in detail in the Example section. The visible platform task was used to confirm that animals have the ability to perform the procedural components of water-escape maze testing, including the visual and motoric capacities necessary to swim towards and climb onto a platform.

For human subjects, nonlimiting example of various neurologic exams in a patient with a suspected dementia include “Wechlser” Memory Scales test, Halstead-Reitan Battery, Trails A and B, Boston Naming Test, Benton Visual Retention Test or Graham-Kendall Memory-for-Designs, Rey-Osterrieth Complex Figure Test, Controlled Oral Word Association Test, tests for left visual neglect, Folstein's Mini-Mental State Exam (MMSE). One or more of these tests may be taken before, during or after the period of treatment characterized by administering a pharmaceutical composition comprising a p38 MAPK inhibitor. In one embodiment, the pharmaceutical composition comprises a compound having a structure or derivative thereof as follows:

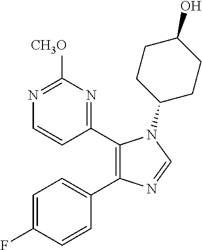

### EXAMPLES

The following examples illustrate certain aspects of the invention. It is to be understood, however, that these examples are provided by way of illustration only, and nothing therein should be deemed a limitation upon the overall scope of the invention.

### Example 1

### p38 MAPK Inhibition Enhances Short Term Memory But Impairs Long Term Memory

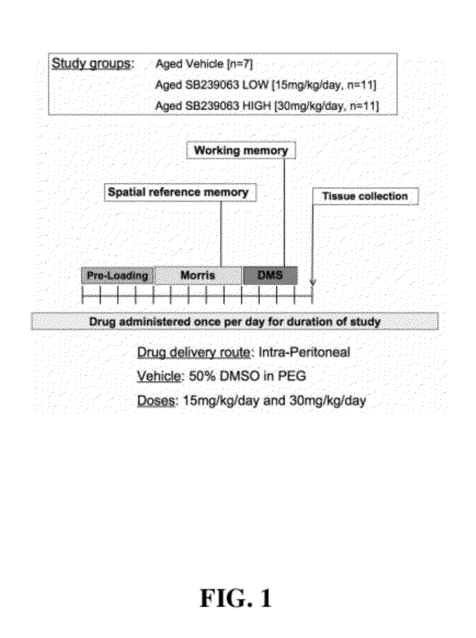

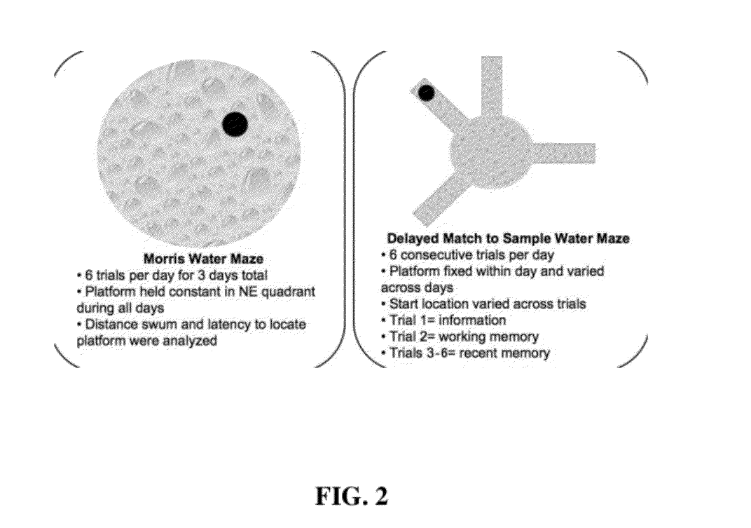

The role of SB239063 on spatial memory performance was tested in the aged rat model. Seventeen month old male Fischer-344 rats received vehicle (50% dimethyl sulfoxide in polyethylene glycol, DMSO-PEG, n=7) or 1 of 2 doses (15 or 30 mg/kg, n=11 per dose) of the p38 MAP Kinase inhibitor SB239063 via daily intraperitoneal injection. Dosing was initiated three days prior to testing and continued throughout the experiment until sacrifice (FIG. 1). Rats were tested on a maze battery including the spatial reference memory Morris maze (MM), and a delay match to sample task (DMS) where the spatial location of the platform needed to be updated on a daily basis, thereby evaluating short term memory (FIG. 2).

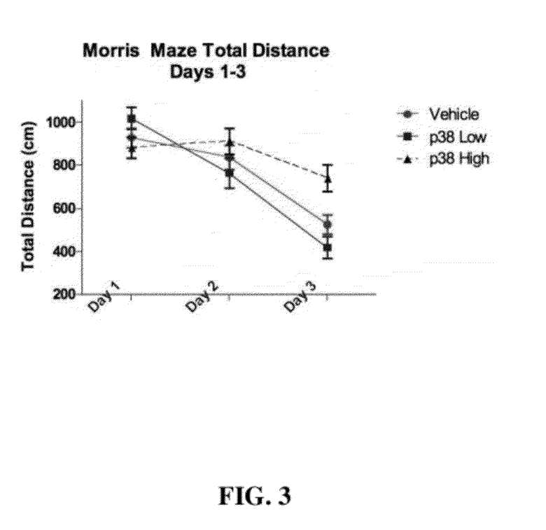

Spatial Reference Memory—Morris Water Maze: For the Morris Water Maze, 6 trials per day for 3 days were carried out. The platform was held constant in NE quadrant during all days, and the distance swam and latency to locate the platform were observed and analyzed among the rats receiving vehicle only (n=7), rats receiving high dose of SB239063 (30 mg/kg; n=10) and rats receiving low dose of SB239063 (15 mg/kg; n=10). All groups localized to the platformed quadrant during the probe trial by the last day of testing (p<0.005). Mean (±Standard Error) swim distance across all days of testing for all rats are depicted in FIG. 3. The swim distance for the Vehicle group is F(1,7)=11.918, (p=0.011) and the SB239063 low dose group is F(1,10)=19.253, (p=0.001), each of which showed a significant linear trend for distance scores across days. In contrast, the SB239063 high dose group with F(1,10)=1.531 (p=0.244) did not show a linear trend for its distance scores across the trial days. In addition, the vehicle-only and low-dose groups improved performance across days but the high-dose group did not.

The 30 mg/kg/day dose impaired MM spatial reference memory performance, while the vehicle and low dose drug treatment groups exhibited significant learning in the MM test as days progressed. This resulted in the 30 mg/kg/day dose exhibiting impaired performance by the lattermost testing day relative to the two other groups (p=0.03) (FIG. 3). These data suggest that p38 MAPK inhibition influences cognitive processing, and can enhance short term memory, but impair long term memory.

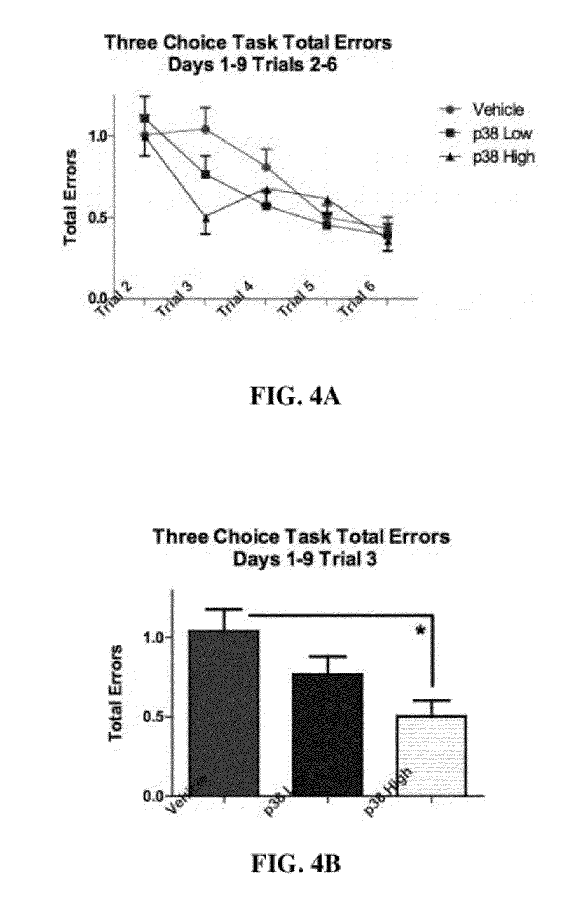

Working Memory—Delay Match to Sample: Rats receiving the high dose of SB239063 at 30 mg/kg/day showed enhanced performance on DMS compared to vehicle-treated animals, with improvements specific to faster learning of the new platform location (p=0.01) (FIG. 4A). Conversely, this high dose impaired Morris Water Maze spatial reference memory performance, whereby the vehicle and low dose drug treatment groups exhibited significant learning as days progressed. There was a significant Trial×Treatment Interaction in the process, and the swim distance F(5,10)=2.298 (p=0.0151). The high-dose receiving group learned the task faster, indicated by committing fewer errors compared to vehicle-only rats on trial 3 (FIG. 4B). The Omnibus ANOVA value for this observation is p=0.036; and the Fisher post hoc value p for High-dose versus Vehicle is 0.011, and thus the inhibition of p38 MAPK improves cognition in the aged rodent is statistically significant. However, the high dose of SB239063 at the 30 mg/kg/day dose exhibited impaired performance by the lattermost testing day relative to the other groups (p=0.03).

Whole Hippocampus Gene Expression Profiling: Gene expression profiling was performed on total RNA isolated from the entire grossly dissected left hippocampus from each individual animal that was vehicle-only, low- or high- dose SB239063 treated rats in the study. At sacrifice, the left hippocampus was collected and the total RNA fraction isolated for gene expression profiling using the Illumina RatRef-12 Expression BeadChip arrays. At a significance of p<0.05 and fold-change cut-off >1.5, there were no differentially expressed transcripts between the groups identified. Therefore, the mechanism for short term memory by inhibition of p38 MAPK does not involve significant changes at the transcriptional level of p38 MAPK down stream genes.

What is claimed is:

1. A method of enhancing short term memory performance comprising the step of:
administering a therapeutically effective dose of a pharmaceutical composition to a subject, wherein said pharmaceutical composition comprises at least one pharmaceutically acceptable carrier and a p38 mitogen activated protein kinase (MAPK) inhibitor.

2. The method of claim 1, wherein the p38 MAPK inhibitor comprises a molecule having a structure or a pharmaceutically acceptable salt thereof as follows:

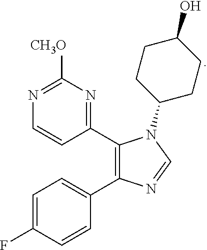

3. The method of claim 1, wherein the short term memory performance is associated with to one or more conditions selected from the group consisting of aging, Alzheimer's disease, amyotrophic lateral sclerosis (ALS), Parkinson's disease, alcoholism or alcohol withdrawal, and Huntington's disease.

4. The method of claim 1, wherein the short term memory impairment is related to Alzheimer's disease.

5. The method of claim 1, wherein the short term memory impairment is related to Alzheimer's disease is Parkinson's disease.

6. The method of claim 1, wherein the effective dose of the pharmaceutical composition comprises a p38 MAPK inhibitor at a range between about 0 to 30 mg/kg (body weight), preferably between about 10 and 20 mg/kg, more preferably at 15 mg/kg; wherein the p38 MAPK inhibitor comprises a molecule having a structure or a pharmaceutically acceptable salt thereof as follows:

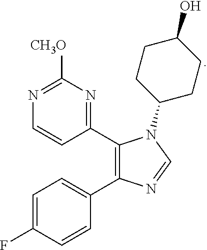

7. The method of claim 1, wherein the effective dose of the pharmaceutical composition comprises a p38 MAPK inhibitor at a range between about 0 to 50 mg/kg (body weight), preferably between about 10 and 40 mg/kg, more preferably at 30 mg/kg; wherein the p38 MAPK inhibitor comprises a molecule having a structure or a pharmaceutically acceptable salt thereof as follows:

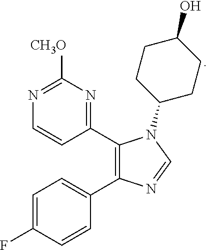

8. The method of claim 1, further comprising the step of administering a second treatment modality to the subject.

9. The method of claim 8, wherein the second treatment modality comprises administering a second pharmaceutical composition to the subject.

10. The method of claim 9 wherein the second pharmaceutical composition is administered concurrently with the first pharmaceutical composition.

11. A method of enhancing spatial reference memory performance comprising the step of:
administering a therapeutically effective dose of a pharmaceutical composition to a subject, wherein said pharmaceutical composition comprises at least one pharmaceutically acceptable carrier and a p38 mitogen activated protein kinase (MAPK) inhibitor.

12. The method of claim 11, wherein the p38 MAPK inhibitor comprises a molecule having a structure or a pharmaceutically acceptable salt thereof as follows:

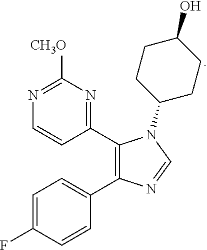

13. The method of claim 11, wherein the spatial reference memory impairment is related to one or more conditions selected from the group consisting of aging, Alzheimer's disease, amyotrophic lateral sclerosis (ALS), Parkinson's disease, alcoholism or alcohol withdrawal and Huntington's disease.

14. The method of claim 14, wherein the spatial reference memory impairment is related to Alzheimer' s disease.

15. The method of claim 14, wherein the spatial reference memory impairment is related to Alzheimer' s disease is Parkinson's disease.

16. The method of claim 11, wherein the effective dose of the pharmaceutical composition comprises a p38 MAPK inhibitor at a range between about 0 to 30 mg/kg (body weight), preferably between about 10 and 20 mg/kg, more preferably at 15 mg/kg; wherein the p38 MAPK inhibitor comprises a molecule having a structure or a derivative thereof as follows:

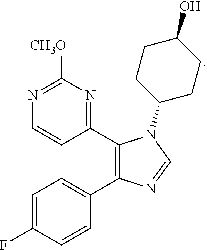

17. The method of claim 11, further comprising the step of administering a second treatment modality to the subject.

18. The method of claim 17, wherein the second treatment modality comprises administering a second pharmaceutical composition to the subject.

19. The method of claim 18 wherein the second pharmaceutical composition is administered concurrently with the first pharmaceutical composition.
